(19) United States
(12) Patent Application Publication
(10) Pub. No.: US 2024/0189562 A1
(43) Pub. Date: Jun. 13, 2024

(54) Skin Disinfectant Wipe

(71) Applicant: Becton, Dickinson and Company, Franklin Lakes, NJ (US)

(72) Inventors: Shishir Prasad, Ramsey, NJ (US); Praveen Nalawade, Belagavi (IN)

(21) Appl. No.: 18/443,511

(22) Filed: Feb. 16, 2024

Related U.S. Application Data
(63) Continuation of application No. PCT/US22/40446, filed on Aug. 16, 2022.
(60) Provisional application No. 63/234,102, filed on Aug. 17, 2021.

(51) Int. Cl. A61M 35/00; A61B 90/80; A61L 2/00

(52) U.S. Cl. CPC A61M 35/006; A61B 90/80; A61L 2/0088

(57) ABSTRACT

A skin preparation device for applying an antiseptic composition to skin of a patient that includes an applicator configured to absorb the antiseptic composition, the applicator comprising a top surface and a bottom surface, the bottom surface configured to apply the antiseptic composition to the skin of the patient, and a gripping portion configured to provide a portion of the skin preparation device that is gripped by a user when using the skin preparation device.

### CROSS-REFERENCE TO RELATED APPLICATIONS

This application is a Bypass Continuation Application of International Application No. PCT/US2022/040446 filed Aug. 16, 2022, and claims priority to United States Provisional Application Ser. No. 63/234,102, filed Aug. 17, 2021, entitled “Skin Disinfectant Wipe”, the entire disclosures of which are hereby incorporated by reference in their entireties.

### BACKGROUND OF THE INVENTION

### Field of the Invention

The present disclosure is directed to a skin disinfectant wipe and, more particularly, is directed to a skin disinfectant wipe that reduces or eliminates contact between a user's skin surface and a patient's skin surface when applying the disinfectant to the patient's skin surface.

### Description of Related Art

Proper cleansing and disinfecting of a patient's skin, for example but not limited to, preparing for injection or aspiration through skin, or a surgery site is crucial to reduce the likelihood of adverse events, including infection, during and following the intervention. However, for many reasons ranging from pressure to rapidly prepare a patient for these procedures to distractions inherent in the environment, many clinicians and other staff do not engage in proper cleansing and disinfecting techniques, for example such as the suggested guidelines of 30 seconds of cleansing, followed by 30 seconds of drying for dry skin areas.

Further, a current method for patient skin preparation is dominated by the use of disinfectant wipes. These disinfectant wipes tend to lead to accidental touching of the patient's skin by the user applying the disinfectant wipe and improper aseptic technique, which both lead to contamination and increased rate of infection on the patient's skin surface.

### SUMMARY OF THE INVENTION

In view of the foregoing disclosure, there is a current need in the art for a disinfectant wipe that reduces or eliminates contact between a user's skin surface and a patient's skin surface when applying the disinfectant to the patient's skin surface. A further need exists in the art for a disinfectant wipe that reduces or eliminates contact between a user's skin surface and a patient's skin surface when applying the disinfectant to the patient's skin surface and remains cost-effective for mass production.

In some non-limiting embodiments and aspects of the present disclosure, a skin preparation device for applying an antiseptic composition to skin of a patient may include an applicator configured to absorb the antiseptic composition, the applicator comprising a top surface and a bottom surface, the bottom surface configured to apply the antiseptic composition to the skin of the patient, and a gripping portion configured to provide a portion of the skin preparation device that is gripped by a user when using the skin preparation device.

In some non-limiting embodiments and aspects of the present disclosure, the applicator and the gripping portion may be made of the same material. The gripping portion may be a ring-shaped structure. The gripping portion may be formed from opposing ends of the skin preparation device that are bent away from the applicator. The applicator and gripping portion may be formed from a single piece of material.

### BRIEF DESCRIPTION OF THE DRAWINGS

FIG. 1 is a perspective view of a skin preparation device according to a non-limiting embodiment or aspect of the present disclosure,

FIG. 2 is a side view of the skin preparation device of FIG. 1,

FIG. 3 is a perspective view of the skin preparation device of FIG. 1 in a compressed state,

FIG. 4 is a side view of the compressed skin preparation device of FIG. 3,

FIG. 5 is a perspective view of a packaging to hold the skin preparation device of FIG. 1 according to one non-limiting embodiment or aspect of the present disclosure,

FIG. 6 is a perspective view of a skin preparation device according to a non-limiting embodiment or aspect of the present disclosure,

FIG. 7 is a side view of the skin preparation device of FIG. 6,

FIG. 8 is a perspective view of the skin preparation device of FIG. 6 in a compressed state,

FIG. 9 is a side view of the compressed skin preparation device of FIG. 8,

FIG. 10 is a perspective view of a skin preparation device according to a non-limiting embodiment or aspect of the present disclosure,

FIG. 11 is a side view of the skin preparation device of FIG. 10,

FIG. 12 is a perspective view of the skin preparation device of FIG. 10 in a compressed state,

FIG. 13 is a side view of the compressed skin preparation device of FIG. 12,

FIG. 14 is a perspective view of a packaging for holding a skin preparation device according to a non-limiting embodiment or aspect of the present disclosure,

FIG. 15 is a perspective view of the skin preparation device of FIG. 6 with a tip of a holding portion bent over,

FIG. 16 is a side view of the compressed skin preparation device of FIG. 15,

FIG. 17 is a perspective view of the packaging of FIG. 14 with a tear-off portion removed,

FIG. 18 is a perspective view of the skin preparation device of FIG. 6 with the tip of the holding portion extended, and

FIG. 19 is a side view of the extended skin preparation device of FIG. 18.

### DESCRIPTION OF THE DISCLOSURE

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

Provided herein is a device for applying an antiseptic composition to skin of a patient, for example for preparing an area of the patient's skin for example, for injection or aspiration through skin, or a surgical intervention. As used herein, the term “patient” is any animal, including humans, and a “human patient” is any human. As used herein, the term “antiseptic composition” is any composition that prevents or inhibits the growth of one or more microorganisms on the patient's skin surface.

As used herein, the term “surgical intervention” means any percutaneous treatment (e.g., catheterization, angioplasty, needle biopsy, and the like), open surgery, laparoscopic surgery, and/or minimally-invasive surgery that involves puncturing the skin or creating one or more incisions of varying size in the skin of the patient.

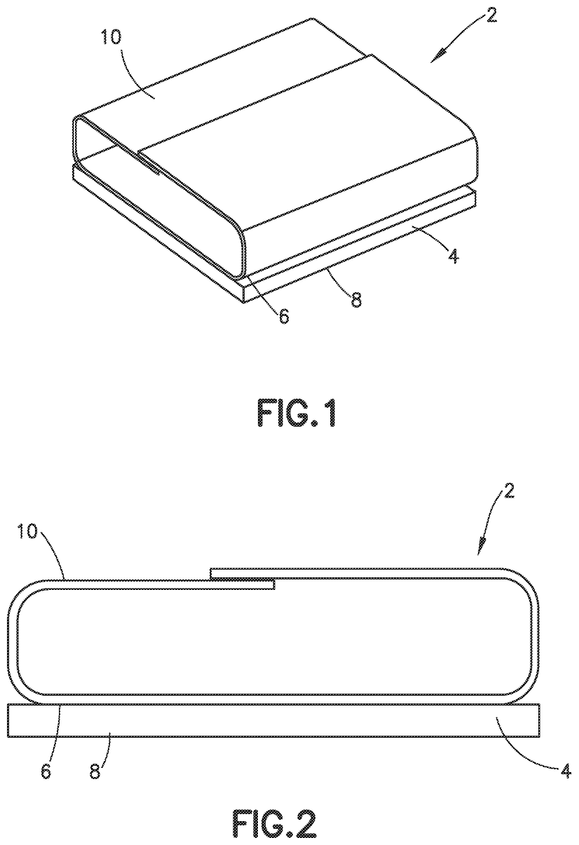

In some non-limiting embodiments or aspects, as shown in FIGS. 1-4, a skin preparation device 2 according to the present disclosure is shown and described in detail. The skin preparation device 2 may include an applicator 4, such as a sponge or other porous, absorbent material that is configured to hold an antiseptic composition, and allows a user to apply the antiseptic composition to the patient's skin at the site of the surgical intervention and, optionally, one or more areas adjacent to the site of the surgical intervention. The applicator 4 can be a natural sponge, a synthetic sponge including, for example and without limitation, a polyurethane, a polyester, and/or a vegetal cellulose, or other suitable material, so long as the material is capable of absorbing and/or dispensing the antiseptic composition. The applicator 4 may have a top surface 6 and a bottom surface 8. Either surface can be used to apply the antiseptic composition to the patient's skin; however, for simplicity, the device 2 will be described herein with reference to the bottom surface 8 as the surface that is configured to come into contact with the patient's skin. In one example, the applicator 4 may be a disinfectant wipe.

In non-limiting embodiments or aspects, the antiseptic composition includes one or more alcohols, such as ethyl alcohol, propyl alcohol, isopropyl alcohol, n-propanol, and/or mixtures thereof. In non-limiting embodiments or aspects, the antiseptic composition includes one or more non-alcohol based compounds, such as iodine, para-chloro-meta-xylenol, bis-biguanides such as chlorhexidine gluconate (CHG), chlorhexidine diacetate or quaterium class compounds such as benzethonium chloride, benzalkonium chloride, chloroxylenol, triclosan, hexachlorophenes, octenidine, diazolidinyl urea, methyl chloro isothiazoline, methyl isothiazoline, triclosan, and/or mixtures thereof. In non-limiting embodiments or aspects, the antiseptic composition includes a mixture of any of the aforementioned, including mixtures of alcohol and non-alcohol based compounds. In non-limiting embodiments or aspects, the antiseptic composition includes CHG and an alcohol, for example isopropyl alcohol. In non-limiting embodiments or aspects, the antiseptic composition includes about 2% (w/v) CHG and about 70% (v/v) isopropyl alcohol.

In non-limiting embodiments or aspects, the antiseptic composition is effective against one or more microorganisms, such as bacteria, viruses, and/or fungi. In non-limiting embodiments or aspects, the microorganism is one or more of coagulase-negative staphylococci, Staphylococcus aureus (including methicillin-resistant S. aureus), Enterococcus spp. (including vancomycin-resistant Enterococci, such as E. faecium), Candida spp., Escherichia coli (including extended-spectrum cephalosporin resistant E. coli and carbanpenem-resistant E. coli), Clostridium difficile, Pseudomonas aeruginosa (including carbapenem-resistant P. aeruginosa), Klebsiella pneumoniae (including extended-spectrum cephalosporin-resistant K. pneumoniae and carbapenem-resistant K. pneumoniae), Enterobacter spp., Acinetobacter spp. (including Acinetobacter baumannii), and Klebsiella oxytoca.

In non-limiting embodiments or aspects, the skin preparation device 2 may include a holding member 10 to allow a user to apply the antiseptic composition to the skin of a patient, while maintaining aseptic technique (e.g., by not directly contacting the applicator or the patient's skin surface). In one example, the holding member 10 may be a looped member that allows a user to insert his/her finger therethrough to use the skin preparation device 2 or to permit a user to grip the holding member 10 to use the skin preparation device 2. In one example, the applicator 4 and the holding member 10 may be made of the same material. The holding member 10 can be a natural sponge, a synthetic sponge including, for example and without limitation, a polyurethane, a polyester, and/or a vegetal cellulose, or other suitable material, so long as the material is capable of absorbing and/or dispensing the antiseptic composition. By using the same material for both the applicator 4 and the holding member 10, the skin preparation device 2 ensures that both the user's skin surface and the patient's skin surface are disinfected. Further, the holding member 10 will cover the user's finger(s) with the material so that, in the event the user's finger(s) touch the patient's skin surface, the material contacts the patient's skin surface instead. In one example, the holding member 10 may be a monolithic piece of material that defines a passageway therethough to receive the user's finger(s). In another example, the holding member 10 may be a piece of material that includes ends bonded to one another to form the passageway to receive the user's finger(s). In one example of the present disclosure, the material of the holding member 10 may be thinner than the material of the applicator 4.

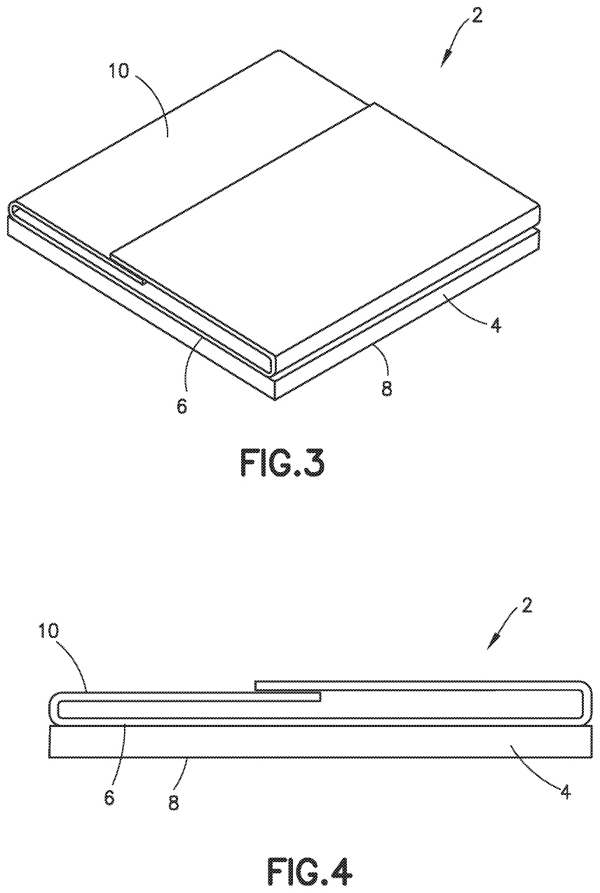

As shown in FIGS. 3-5, according to non-limiting embodiments or aspects of the present disclosure, the skin preparation device 2 may be provided in a packaging 12. The packaging 12 may be made of at least two pieces of material that receive the skin preparation device 2 therebetween. The packaging 12 may be opened by pulling a tab 14 that causes the two pieces of material to be pulled apart. As shown in FIG. 4, when the skin preparation device 2 is held in the packaging 12, the holding member 10 may be flattened to fit within the packaging 12. After the skin preparation device 2 has been removed from the packaging 12, the holding member 10 may spring into a use position or may be pulled in to a use position by the user so that a user may insert his/her finger through the holding member 10.

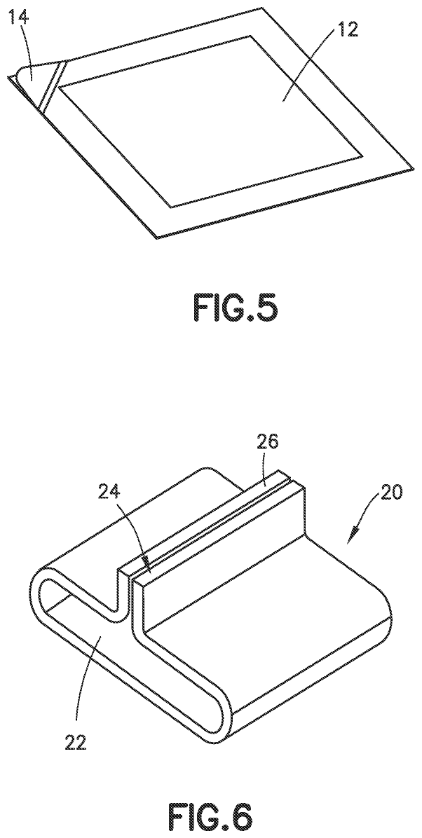

With reference to FIGS. 6-9, according to some non-limiting embodiments and aspects, a skin preparation device 20 is shown and described in detail. The skin preparation device 20 may include an applicator portion 22 and a handle portion 24. In one example, the applicator portion 22 and the handle portion 24 are formed as a monolithic structure and are formed integral with one another from a single piece of material. In other examples, the applicator portion 22 and the handle portion 24 may be two pieces of material operatively connected to one another. The applicator portion 22 and the handle portion 24 may form a ring-like structure that has a passageway defined therethrough.

The applicator portion 22 can be a natural sponge, a synthetic sponge including, for example and without limitation, a polyurethane, a polyester, and/or a vegetal cellulose, or other suitable material, so long as the material is capable of absorbing and/or dispensing the antiseptic composition. In one example, the handle portion 24 may be made of the same material as the applicator portion 22. In one example, the applicator portion 22 may be a disinfectant wipe.

In non-limiting embodiments or aspects, the antiseptic composition includes one or more alcohols, such as ethyl alcohol, propyl alcohol, isopropyl alcohol, n-propanol, and/or mixtures thereof. In non-limiting embodiments or aspects, the antiseptic composition includes one or more non-alcohol based compounds, such as iodine, para-chloro-meta-xylenol, bis-biguanides such as chlorhexidine gluconate (CHG), chlorhexidine diacetate or quaterium class compounds such as benzethonium chloride, benzalkonium chloride, chloroxylenol, triclosan, hexachlorophenes, octenidine, diazolidinyl urea, methyl chloro isothiazoline, methyl isothiazoline, triclosan, and/or mixtures thereof. In non-limiting embodiments or aspects, the antiseptic composition includes a mixture of any of the aforementioned, including mixtures of alcohol and non-alcohol based compounds. In non-limiting embodiments or aspects, the antiseptic composition includes CHG and an alcohol, for example isopropyl alcohol. In non-limiting embodiments or aspects, the antiseptic composition includes about 2% (w/v) CHG and about 70% (v/v) isopropyl alcohol.

In non-limiting embodiments or aspects, the antiseptic composition is effective against one or more microorganisms, such as bacteria, viruses, and/or fungi. In non-limiting embodiments or aspects, the microorganism is one or more of coagulase-negative staphylococci, Staphylococcus aureus (including methicillin-resistant S. aureus), Enterococcus spp. (including vancomycin-resistant Enterococci, such as E. faecium), Candida spp., Escherichia coli (including extended-spectrum cephalosporin resistant E. coli and carbanpenem-resistant E. coli), Clostridium difficile, Pseudomonas aeruginosa (including carbapenem-resistant P. aeruginosa), Klebsiella pneumoniae (including extended-spectrum cephalosporin-resistant K. pneumoniae and carbapenem-resistant K. pneumoniae), Enterobacter spp., Acinetobacter spp. (including Acinetobacter baumannii), and Klebsiella oxytoca.

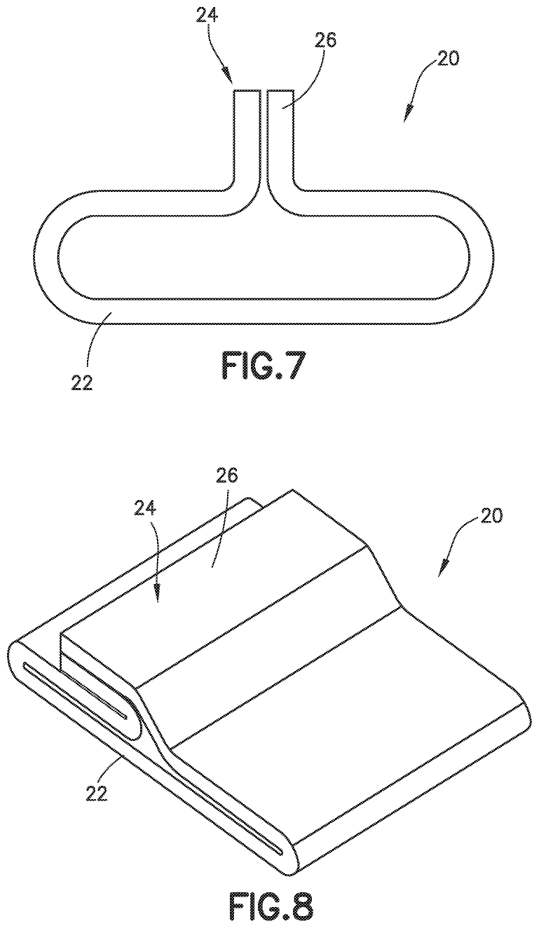

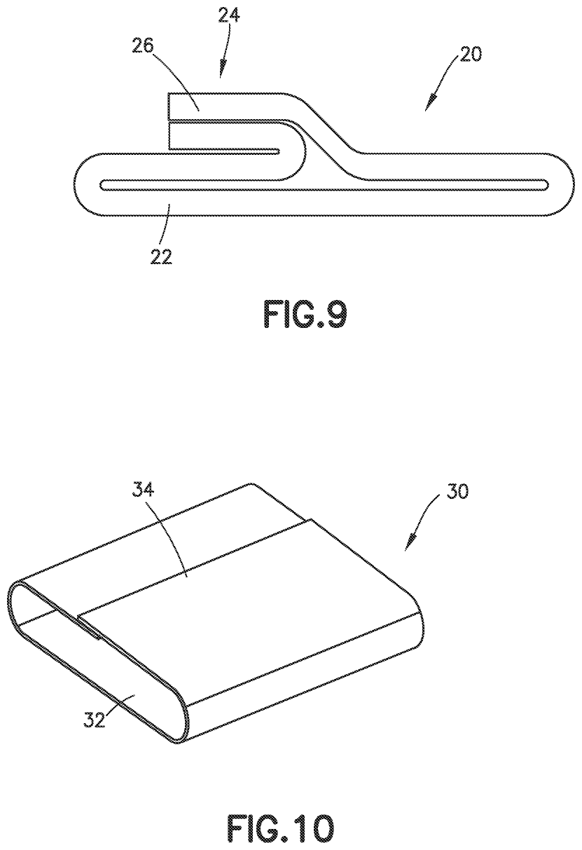

In some non-limiting embodiments and aspects, the handle portion 24 may include the two opposing ends of the skin preparation device 20. The handle portion 24 may bend upwardly from the applicator portion 22 to form gripping portions for the user to grasp when using the skin preparation device 20. In one example, the opposing ends of the skin preparation device 20 may be bonded together to form the handle portion 24. In some examples, the opposing ends of the skin preparation device 20 may be adhesively bonded or heat bonded. In order to signify to the user the location of the handle portion 24, the handle portion 24 may be colored differently from the applicator portion 22. In another example, the handle portion 24 may include written instructions or pictorials to indicate to the user that the handle portion 24 should be grasped. As shown in FIGS. 7 and 9, in some non-limiting embodiments or aspects, the handle portion 24 may be folded over the applicator portion 22 when held in a packaging (not shown) to reduce the overall size of the skin preparation device 20 when held in the packaging. When in use, the user may squeeze the handle portion 24 between his/her thumb and index finger to grip the skin preparation device 20.

With reference to FIGS. 10-13, according to some non-limiting embodiments and aspects, a skin preparation device 30 is shown and described in detail. The skin preparation device 30 may include an applicator portion 32 and a clasping portion 34. In one example, the applicator portion 32 and the clasping portion 34 are formed as a monolithic structure and are formed integral with one another from a single piece of material. In other examples, the applicator portion 32 and the clasping portion 34 may be two pieces of material operatively connected to one another. In some examples, the opposing ends of the skin preparation device 30 may be adhesively bonded or heat bonded together. The applicator portion 32 and the clasping portion 34 may form a ring-like structure that has a passageway defined therethrough, in which the user can insert his/her finger to use the skin preparation device 30.

The applicator portion 32 can be a natural sponge, a synthetic sponge including, for example and without limitation, a polyurethane, a polyester, and/or a vegetal cellulose, or other suitable material, so long as the material is capable of absorbing and/or dispensing the antiseptic composition. In one example, the clasping portion 34 may be made of the same material as the applicator portion 32. In one example, the applicator portion 32 may be a disinfectant wipe.

In non-limiting embodiments or aspects, the antiseptic composition includes one or more alcohols, such as ethyl alcohol, propyl alcohol, isopropyl alcohol, n-propanol, and/or mixtures thereof. In non-limiting embodiments or aspects, the antiseptic composition includes one or more non-alcohol based compounds, such as iodine, para-chloro-meta-xylenol, bis-biguanides such as chlorhexidine gluconate (CHG), chlorhexidine diacetate or quaterium class compounds such as benzethonium chloride, benzalkonium chloride, chloroxylenol, triclosan, hexachlorophenes, octenidine, diazolidinyl urea, methyl chloro isothiazoline, methyl isothiazoline, triclosan, and/or mixtures thereof. In non-limiting embodiments or aspects, the antiseptic composition includes a mixture of any of the aforementioned, including mixtures of alcohol and non-alcohol based compounds. In non-limiting embodiments or aspects, the antiseptic composition includes CHG and an alcohol, for example isopropyl alcohol. In non-limiting embodiments or aspects, the antiseptic composition includes about 2% (w/v) CHG and about 70% (v/v) isopropyl alcohol.

In non-limiting embodiments or aspects, the antiseptic composition is effective against one or more microorganisms, such as bacteria, viruses, and/or fungi. In non-limiting embodiments or aspects, the microorganism is one or more of coagulase-negative staphylococci, Staphylococcus aureus (including methicillin-resistant S. aureus), Enterococcus spp. (including vancomycin-resistant Enterococci, such as E. faecium), Candida spp., Escherichia coli (including extended-spectrum cephalosporin resistant E. coli and carbanpenem-resistant E. coli), Clostridium difficile, Pseudomonas aeruginosa (including carbapenem-resistant P. aeruginosa), Klebsiella pneumoniae (including extended-spectrum cephalosporin-resistant K. pneumoniae and carbapenem-resistant K. pneumoniae), Enterobacter spp., Acinetobacter spp. (including Acinetobacter baumannii), and Klebsiella oxytoca.

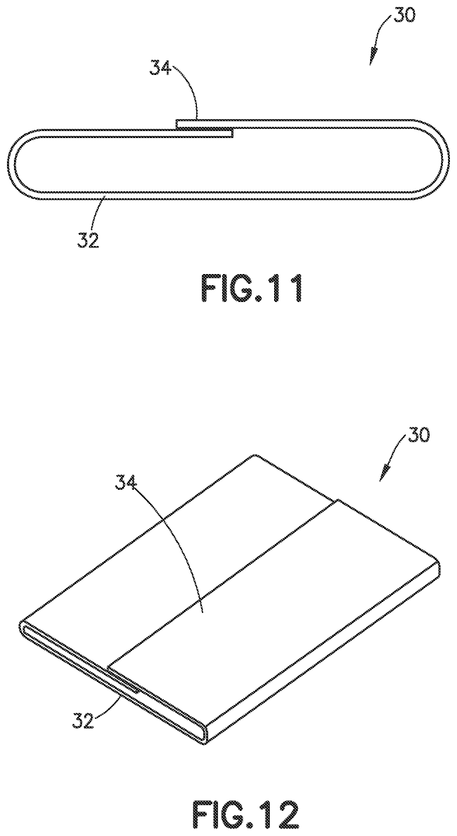

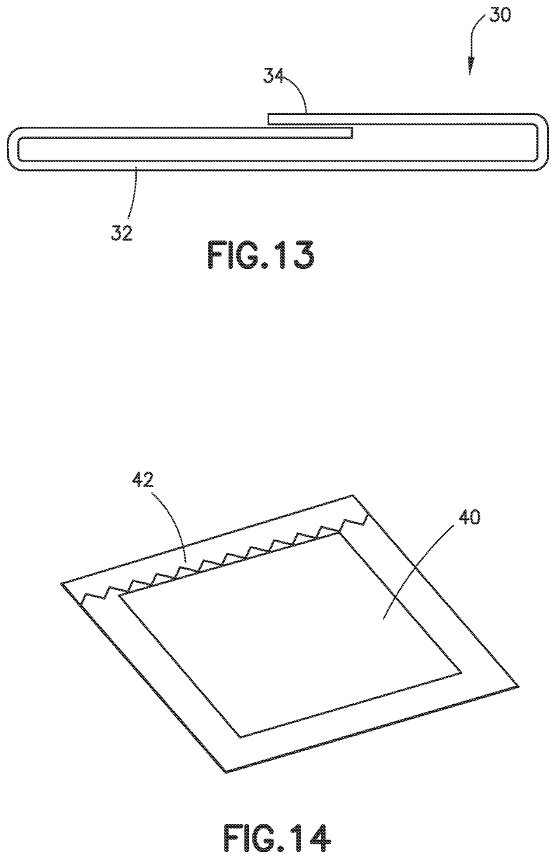

In one example, the opposing ends of the skin preparation device 30 may be bonded together to form the clasping portion 34. As shown in FIGS. 11 and 13, according to some non-limiting embodiments or aspects, the skin preparation device 30 may be folded to deflate the ring-like structure to reduce the overall size of the skin preparation device 30 when packaging the skin preparation device 30.

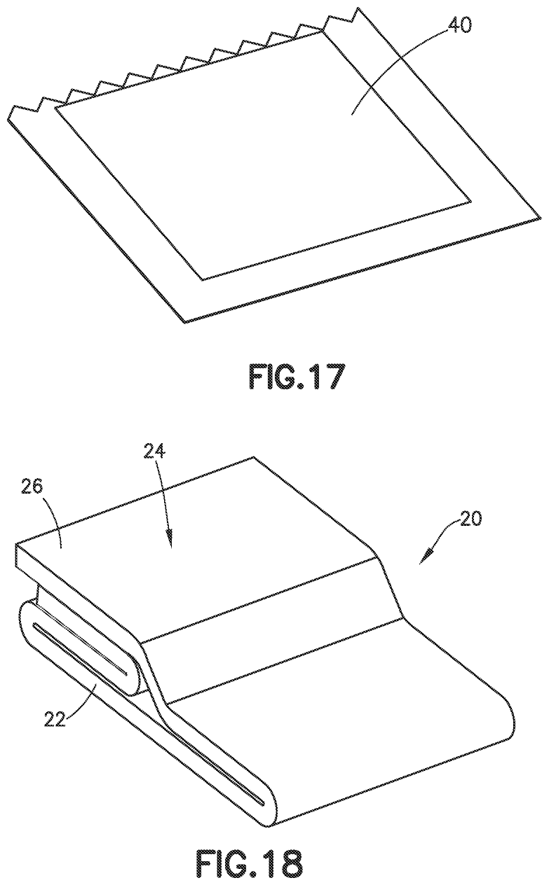

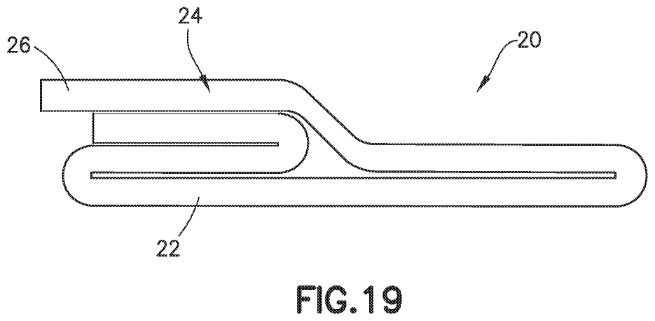

With reference to FIGS. 14-19, according to some non-limiting embodiments and aspects, a packaging 40 for the skin preparation device 20 is shown and described. The packaging 40 may define a cavity to receive the skin preparation device 20. A tear-off portion 42 may be provided on one end of the packaging 40 to permit a user to tear off a portion of the packaging 40 to remove the skin preparation device 20 therefrom. The tear-off portion 42 may be precut to allow for an easier removal of the tear-off portion 42 from the packaging 40.

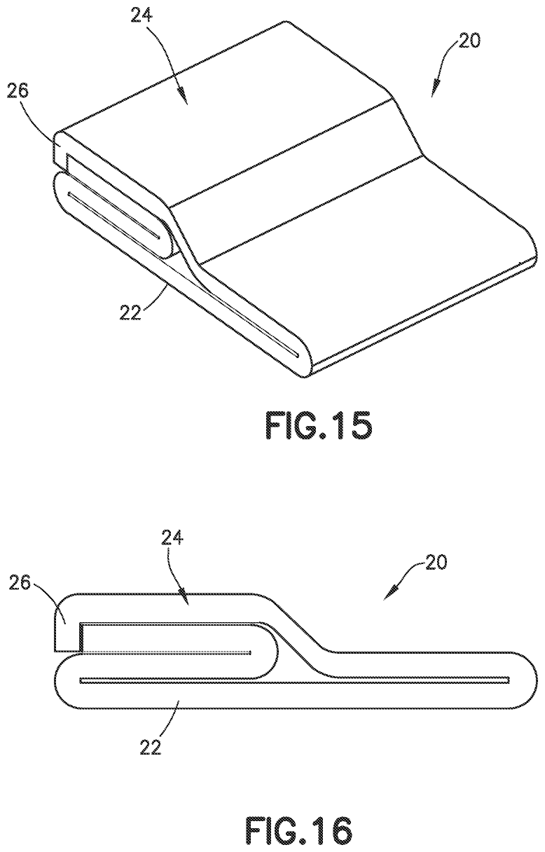

As shown in FIGS. 15 and 16, according to some non-limiting embodiments or aspects, the handle portion 24 of the skin preparation device 20 may be folded over onto the applicator portion 22 to reduce the overall size of the skin preparation device 20. The handle portion 24 may be folded towards the tear-off portion 42 of the packaging 40 so that, after the tear-off portion 42 has been removed, the user may grip the handle portion 24 and pull the skin preparation device 20 from the packaging 40 without contaminating the applicator portion 22. In one example, a tip 26 of the handle portion 24 may be bent over the remaining portion of the handle portion 24. After the tear-off portion 42 has been removed from the packaging 40, the tip 26 may flex away from the handle portion 24 and out of the packaging 40 for easy gripping by the user.

The skin preparation devices 2, 20, 30 of the present disclosure enable aseptic non-touch skin preparation for intravenous (IV) line insertion, especially for peripheral intravenous catheters (PIVC). Furthermore, the skin preparation devices 2, 20, 30 and the packaging 40 result in low cost device and adequately treat patients in a price-conscious market. The skin preparation devices 2, 20, 30 provide improved gripping arrangements for the disinfectant wipes to avoid accidental touching of the active surface of the wipe intended for skin preparation. In addition, the packaging 40 is also be optimized to aid in the aseptic non-touch technique. The present disclosure adds minimal cost to the current low cost wipes in terms of providing handles for aseptic non-touch holding of the wipes and adjusting rigidity of the wipe for easy grabbing of the handle.

The current disclosure utilizes the device design (creatively making the gripping portion of the same material as the wipe for aseptic use of the skin preparation), material science (optimized rigidity of the wipes for the gripping portion for easy access and optimized porosity of the wipe to retain the required amount of disinfectant liquid and at the same time not dripping when taken out from the packaging), and packaging features (packaging direction for opening the packaging with the gripping portion directed towards the user and tear pattern perpendicular to the gripping portion for the gripping portion to spring outwardly when the package is teared to enable easy gripping portion access).

The device material property is one important factor in this disclosure. Optimized rigidity of the wipe material enables the gripping portion to spring out when the packaging is opened and at the same time remain inside the packaging without damaging it over the shelf life. Optimized porosity of the packaging material results in the required amount of disinfectant liquid incorporated into the device and at the same time not dripping when the device is taken out of the packaging over the product shelf life.

The present disclosure provides an improved combination of device design, material properties, and packaging features to address the shortcomings of wipes in skin preparation, i.e. being able to aseptic non-touch application of skin preparation device. A gripping portion made of the same material as the wipe provides cost effective means to incorporate this feature for a price sensitive market.

While the present invention has been described in terms of the above detailed description, those of ordinary skill in the art will understand that alterations may be made within the spirit of the invention.

What is claimed is:

1. A skin preparation device for applying an antiseptic composition to skin of a patient, comprising:
an applicator configured to absorb the antiseptic composition, the applicator comprising a top surface and a bottom surface, the bottom surface configured to apply the antiseptic composition to the skin of the patient; and
a gripping portion configured to provide a portion of the skin preparation device that is gripped by a user when using the skin preparation device.

2. The skin preparation device of claim 1, wherein the applicator and the gripping portion are made of the same material.

3. The skin preparation device of claim 1, wherein the gripping portion is a ring-shaped structure.

4. The skin preparation device of claim 1, wherein the gripping portion is formed from opposing ends of the skin preparation device.

5. The skin preparation device of claim 4, wherein the opposing ends of the skin preparation device are bent away from the applicator.

6. The skin preparation device of claim 1, wherein the applicator and the gripping portion are formed from a single piece of material.

7. The skin preparation device of claim 1, wherein the applicator is at least one of a natural sponge and a synthetic sponge.

8. The skin preparation device of claim 7, wherein the synthetic sponge comprises at least one of a polyurethane, a polyester, and/or a vegetal cellulose.

9. The skin preparation device of claim 1, wherein the antiseptic composition includes one or more alcohols, comprising ethyl alcohol, propyl alcohol, isopropyl alcohol, n-propanol, and/or mixtures thereof.

10. The skin preparation device of claim 1, wherein the antiseptic composition includes one or more non-alcohol based compounds.

11. The skin preparation device of claim 10, wherein the one or more non-alcohol based compounds comprise at least one of iodine, para-chloro-meta-xylenol, bis-biguanides, chlorhexidine gluconate (CHG), chlorhexidine diacetate, a quaterium class compound, benzethonium chloride, benzalkonium chloride, chloroxylenol, triclosan, hexachlorophene, octenidine, diazolidinyl urea, methyl chloro isothiazoline, methyl isothiazoline, triclosan, and/or mixtures thereof.

12. The skin preparation device of claim 1, wherein the antiseptic composition includes a mixture of alcohol and non-alcohol based compounds.

13. The skin preparation device of claim 1, wherein the antiseptic composition includes CHG and an alcohol.

14. The skin preparation device of claim 13, wherein the alcohol is isopropyl alcohol.

15. The skin preparation device of claim 14, wherein the antiseptic composition includes about 2% (w/v) CHG and about 70% (v/v) isopropyl alcohol.

16. A skin preparation device for applying an antiseptic composition to skin of a patient, comprising:
an applicator configured to absorb the antiseptic composition, the applicator comprising a top surface and a bottom surface, the bottom surface configured to apply the antiseptic composition to the skin of the patient; and
a clasping portion, connected with the applicator, wherein the applicator and the clasping portion form a ring-like structure that has a passageway defined therethrough, in which a user can insert a finger to use the skin preparation device.

17. The skin preparation device of claim 16, wherein the applicator and the clasping portion are formed as a monolithic structure.

18. The skin preparation device of claim 16, wherein the applicator and the clasping portion are formed separately and operatively connected to one another.

19. The skin preparation device of claim 16, wherein the antiseptic composition includes one or more alcohols, comprising ethyl alcohol, propyl alcohol, isopropyl alcohol, n-propanol, and/or mixtures thereof.

20. The skin preparation device of claim 16, wherein the one or more non-alcohol based compounds comprise at least one of iodine, para-chloro-meta-xylenol, bis-biguanides, chlorhexidine gluconate (CHG), chlorhexidine diacetate, a quaterium class compound, benzethonium chloride, benzalkonium chloride, chloroxylenol, triclosan, hexachlorophene, octenidine, diazolidinyl urea, methyl chloro isothiazoline, methyl isothiazoline, triclosan, and/or mixtures thereof.
